(19) United States
(12) Patent Application Publication
(10) Pub. No.: US 2009/0156808 A1
(43) Pub. Date: Jun. 18, 2009

(54) PROCESSES FOR THE PREPARATION OF 3,4-SUBSTITUTED-1,2,5-THIADIAZOLES AND INTERMEDIATES THEREOF

(76) Inventors: Alfio Borghese, Rue Granbonpre (IT); Vincenzo Mancuso, Rue Granbonpre (BE); Alain Merschaert, Rue Granbonpre (BE)

Correspondence Address: ELI LILLY & COMPANY, PATENT DIVISION, P.O. BOX 6288, INDIANAPOLIS, IN, 46206-6288, US

(21) Appl. No.: 11/719,552

(22) PCT Filed: Dec. 7, 2005
(86) PCT No.: PCT/US05/44117

§ 371 Date: May 17, 2007

Related U.S. Application Data
(60) Provisional application No. 60/637,526, filed on Dec. 20, 2004.

(51) Int. Cl. C07D 265/30; C07C 255/10; C07D 211/96; C07F 7/10

(52) U.S. Cl. 544/159; 558/436; 556/412; 546/246

(57) ABSTRACT

The present invention relates to intermediate compounds useful for the preparation of 1,2,5-thiadiazole compounds, including unsymmetrically substituted compounds such as 4-(3-secondary amino-2-hydroxy-propoxy)-1,2,5-thiadiazole compounds and azacyclic or azabicyclic 1,2,5-thiadiazole compounds and to processes for making the same.

The present invention relates to intermediate compounds useful for the preparation of 1,2,5-thiadiazole compounds, including unsymmetrically substituted compounds such as 4-(3-secondary amino-2-hydroxy-propoxy)-1,2,5-thiadiazole compounds which are useful as β-adrenergic blocking agents and azacyclic or azabicyclic1,2,5-thiadiazole compounds which are useful in the modulation of a muscarinic chiolinergic receptor. The present invention also relates to processes for making the same.

### BACKGROUND OF THE INVENTION

Thiadiazoles are known, including certain unsymmetrically substituted 1,2,5-thiadiazoles, such as 4-(3-secondary amino-2-hydroxy-propoxy)-1,2,5-thiadiazole compounds described in U.S. Pat. No. 3,655,663, B. K. Wasson et al., J. Med. Chem., 15, pp. 51-655 (1972), and L. M. Weinstock, et al., J. Org. Chem., 41, pp. 3121-3214 (1976); and azacyclic or azabicyclic 1,2,5-thiadiazole compounds described in U.S. Pat. No. 5,665,745 and J. S. Ward et al., J. Med. Chem., 41, pp. 379-392 (1998). Intermediates and processes for preparing such compounds are also known, such as those described in the above mentioned documents and in U.S. Pat. Nos. 5,672,709 and 5,834,458 and PCT Applications WO 97/39753 and WO 98/54151.

These methods show the difficulty in achieving differential substitution at the 3- and 4-positions of a 1,2,5-thiadiazole. The present invention provides intermediates and processes that allow for the facile and efficient introduction of substituents at the 3- and 4-positions, including unsymmetrical substitution.

### SUMMARY OF THE INVENTION

The present invention provides a process for preparing a compound of the formula VI(a)

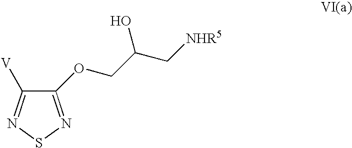

wherein

V is selected from the group consisting of 4-morpholinyl, N-methyl-1-piperazinyl and 1-piperidinyl; and

R⁵ is C3-C6 alkyl;

comprising

(a) reacting a 2-L-2-(R¹, R²-aminothio)imino acetonitrile of formula I

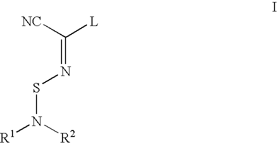

wherein

L is a leaving group;

R¹ and R² are independently selected from the group consisting of hydrogen, C1-C4 alkyl, benzyl optionally substituted with one or more substituents independently selected from the group consisting of halo, C1-C4 alkyl, C1-C4 alkoxy and —CF₃, and —SiRRR wherein each R is independently selected from the group consisting of C₁-C₄ alkyl and phenyl optionally substituted with one or more substituents independently selected from the group consisting of C1-C4 alkyl, and C1-C4 alkoxy;

provided when R¹ is hydrogen then R² is not hydrogen;
or

R¹ and R² are taken together with the nitrogen in which they are attached to form a ring of formula II

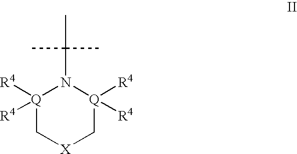

wherein

X is selected from the group consisting of bond, —CH₂—, —S(O)—ₙ, —O— and —NR²¹— wherein R²¹ is selected from the group consisting of C1-C4 alkyl, benzyl and phenyl optionally substituted with one or more substituents independently selected from the group consisting of halo, C1-C4 alkyl and —CF₃;

n is 0, 1 or 2; and

Q is selected from the group consisting of a bond, carbon atom wherein each R⁴ is independently selected from the group consisting of hydrogen and C1-C4 alkyl, and a silicon atom wherein each R⁴ is independently selected from the group consisting of C1-C4 alkyl and phenyl;

with a V—H wherein V is as defined above;

to give the 2-V-2-(R¹, R²-aminothio)imino acetonitrile derivative of formula IV

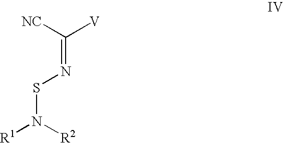

wherein R¹, R² and V are as defined above;

(b) reacting the derivative 2-V-2-(R¹, R²-aminothio)imino acetonitrile with a compound of formula

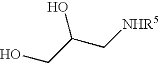

wherein R⁵ is as defined above;

to give a compound of formula VI(a) as described above.

Further, the present invention provides a process for preparing a compound of the formula VI(b)

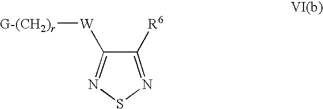

wherein

W is O or S;

R⁶ is selected from the group consisting of —NHR¹⁰, —NR⁷R⁸, —OR⁹, —SR⁹, -Z¹-C3-C7 cycloalkyl, -Z¹-C4-C10 cycloalkylalkyl, —O(CH₂)ₘY, and —S(CH₂)ₘY;

Z¹ is O or S;

R¹⁰ is selected from the group consisting hydrogen and C1-C6 alkyl;

R⁷ and R⁸ are independently selected from the group consisting of hydrogen and C1-C6 alkyl, or

R⁷ and R⁸ taken together with nitrogen to which they are attached form a 4 to 6 membered ring;

R⁹ is selected from the group consisting of C1-C5 alkyl, C2-C5 alkenyl and C2-C5 alkynyl, each of which is optionally substituted with one or more substituents independently selected from the group consisting of halogen, —CF3, —CN, Y, phenyl optionally substituted with one or more substituents independently selected from halogen, —CN, C1-C4 alkyl, C1-C4 alkoxy, —OCF₃ and —CF₃, and phenoxy optionally substituted with one or more substituents independently selected from halogen, C1-C4 alkyl, C1-C4 alkoxy, and —CF₃;

Y is selected from the group consisting of pyridyl, thienyl, furanyl, piperidinyl and benzthienyl;

m is 1, 2, 3, 4, or 5;

r is 0, 1, 2 or 3;

G is selected from the group consisting of

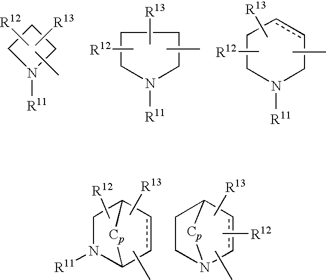

R¹¹ is C1-C5 alkyl;

R¹² and R¹³ are independently selected from the group consisting of hydrogen, C1-C6 alkyl, and C1-C4 alkoxy;

[symbol] is a single or double bond; and

p is 1 or 2;

comprising

(a) reacting a 2-L-2-(R¹, R²-aminothio)imino acetonitrile of formula I

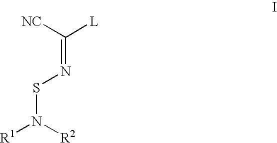

wherein

L, R¹ and R² are defined as above;

with a compound of the formula R⁶—H wherein R⁶ is as defined above;

to give the 2-R⁶-2-(R¹, R²-aminothio)imino acetonitrile derivative of formula III

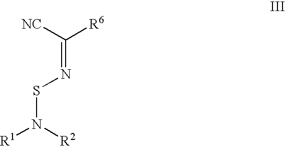

wherein R¹, R², and R⁶ are as defined above;

(b) reacting the 2-R⁶-2-(R¹, R²-aminothio)imino acetonitrile derivative of formula III with a compound of formula G-(CH₂)ᵣ—W—H wherein G, r and W are defined as above, to provide a compound of formula VI(b) as described above.

Further, the present invention provides a compound of formula I

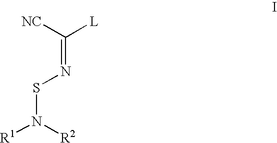

wherein

L is a leaving group;

R¹ and R² are independently selected from the group consisting of hydrogen, C1-C4 alkyl, benzyl optionally substituted with one or more substituents independently selected from the group consisting of halo, C1-C4 alkyl, C1-C4 alkoxy and —CF₃; and —SiRRR wherein each R is independently selected from the group consisting of C1-C4 alkyl, and phenyl optionally substituted with one or more substituents independently selected from the group consisting of C1-C4 alkyl, and C1-C4 alkoxy;

provided when R¹ is hydrogen then R² is not hydrogen;

or

R¹ and R² are taken together with the nitrogen in which they are attached to form a ring of formula II

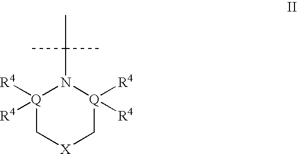

wherein

X is selected from the group consisting of bond, —CH₂—, —S(O)—ₙ, —O— and —NR²—wherein R²¹ is selected from the group consisting of C1-C4 alkyl, benzyl and phenyl optionally substituted with one or more substituents independently selected from the group consisting of halo, C1-C4 alkyl, and —CF₃;

n is 0, 1 or 2;

and

Q is selected from the group consisting of a bond, a carbon atom wherein each R⁴ is independently selected from the group consisting of hydrogen and C1-C4 alkyl; and a silicon atom wherein each R⁴ is independently selected from the group consisting of C1-C4 alkyl and phenyl.

Further, the present invention provides a compound of formula III

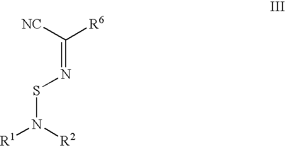

wherein R¹, R² and R⁶ are as described above.

Further, the present invention provides a compound of formula IV

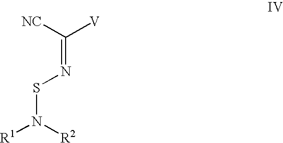

wherein V, R¹ and R² are as described above.

Further the present invention provides a compound of formula VII

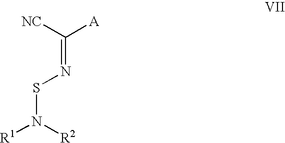

wherein

A is independently selected from the group consisting of

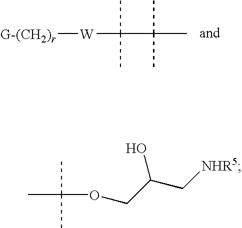

and

R¹, R², G, W, r and R⁵ are as defined above.

### DETAILED DESCRIPTION OF THE INVENTION

As used herein, the following terms have the meanings indicated:

The term “C1-C6 alkyl” refers to a straight or branched alkyl chain having from one to six carbon atoms, and includes methyl, ethyl, propyl, iso-propyl, butyl, iso-butyl, sec-butyl, t-butyl, pentyl, 2-methylpentyl, hexyl and the like.

The term “C3-C6 alkyl” refers to a straight or branched alkyl chain having form three to six carbon atoms, and includes propyl, iso-propyl, butyl, iso-butyl, sec-butyl, t-butyl, pentyl, 2-methylpentyl, hexyl and the like.

The term “C1-C5 alkyl” refers to a straight or branched alkyl chain having from one to five carbon atoms, and includes methyl, ethyl, propyl, iso-propyl, butyl, iso-butyl, sec-butyl, t-butyl, pentyl and the like.

The term “C1-C4 alkyl” refers to a straight or branched alkyl chain having from one to four carbon atoms, and includes methyl, ethyl, propyl, iso-propyl, butyl, iso-butyl, sec-butyl, t-butyl and the like.

The term “C1-C4 alkoxy” refers to straight or branched alkyl chain having from one to four carbon atoms attached to an oxygen atom, and includes methoxy, ethoxy, propoxy, iso-propoxy, butoxy, iso-butoxy, sec-butoxy, t-butoxy, and the like.

The term “C3-C7 cycloalkyl” refers to saturated cyclic alkyl group having from three to seven carbon atoms and includes cyclopropyl, cyclobutyl, cyclopentyl, cyclohexyl, and cycloheptyl.

The term “C4-C10 cycloalkylalkyl” refers to saturated cyclic alkyl group having from four to seven carbon atoms linked to the point of substitution by a divalent unsubstituted saturated straight-chain or branched-chain hydrocarbon radical having at least 1 carbon atom and includes, cyclopropylmethyl, cyclopropyl-2-propyl, cyclobutylethyl, cyclopentylmethyl, cyclohexylmethyl, cycloheptylmethyl and the like.

The term “halogen or halo” refers to chloro, fluoro, bromo or iodo.

The term “C2-C5 alkenyl” refers to a straight or branched alkyl chain having from one to five carbon atoms and one carbon-carbon double bond, and includes ethenyl, propenyl, iso-propenyl, butenyl, iso-butenyl, sec-butenyl, pentenyl and the like.

The term “C2-C5 alkynyl” refers to a straight or branched alkyl chain having from one to five carbon atoms and one carbon-carbon triple bond, and includes 1-propynyl, 2-propynyl, 1-butynyl, 2-butynyl, 1-pentynyl and the like.

The term “leaving group” refers to a chemical group capable of being displaced by a nucleophile. The structure of the leaving group will depend, in part, on the general reaction conditions employed such as structure of the nucleophile, solvent, temperature and time, all within the knowledge and control of the skilled artisan. Examples of leaving groups commonly employed include sulfonyl esters such as trifluoromethylsulfonyl, para-nitrobenzenesulfonyl, para-toluenesulfonyl, methylsulfonyl and the like; carboxyl esters such as trifluoroacetyl, para-nitrobenzoyl, para-methylbenzoyl, acetyl and the like; and halogens such as iodo, bromo, chloro, fluoro and the like.

As is apparent to those skilled in the art, the compounds of the present invention may exist as tautomers. Where tautomers exist, each tautomeric form and mixtures thereof, are contemplated as included in the present invention.

It is understood that compounds of the present invention may exist as stereoisomers. All stereoisomers, and mixtures thereof, are contemplated within the present invention.

As with any group of intermediates some embodiments are preferred in their application. Preferred embodiments for a compound of formula I of the present invention are given below.

Compounds of formula I in which L is halogen are preferred. Compounds in which L is chloro are more preferred.

Compounds of formula I in which R¹ and R² are C1-C4 alkyl group are preferred.

Compounds of formula I in which R¹ and R² are taken together with the nitrogen in which they are attached to form a ring of formula II

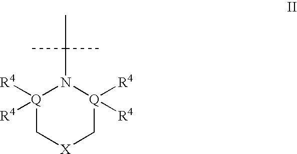

wherein

X is selected from the group consisting of —CH₂— and —O—;

Q is a carbon atom wherein each R⁴ is C1-C4 alkyl or a silicon atom wherein R⁴ is C1-C4 are preferred.

Compounds of formula I in which R¹ and R² are SiRRR wherein each R is independently selected from the group consisting of C1-C4 alkyl are more preferred.

Compounds of formula I, which are given below are even more preferred.

### 2-chloro-2-(N,N-bis(trimethylsilyl)aminothio)imino acetonitrile

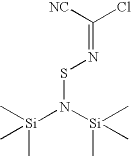

### 2-chloro-2-(4-morpholinylthio)imino acetonitrile

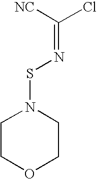

### 2-chloro-2-(2,2,6,6-tetramethyl-1-piperidinylthio)imino acetonitrile

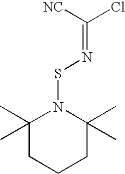

Synthetic procedures are given below. In those procedures it is understood that the reactions may be monitored by known techniques such as chromatography. The reaction products may be isolated by techniques known to the skilled artisan such as quenching the reaction with water, extraction and evaporation and that the products may be purified by techniques well known and appreciated in the art such as distillation, crystallization or chromatography.

A general synthetic procedure is presented in Scheme A. All substituents unless otherwise indicated are as described above.

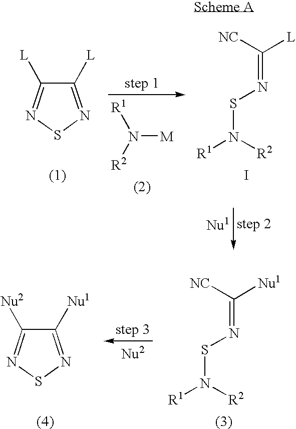

In Scheme A, step 1, for an appropriate compound of formula (1) is reacted with an appropriate metal amide of formula (2) to give a compound of formula I. An appropriate compound of formula (1) is one in which the group L is desired in a compound of formula I. An appropriate compound of formula (2) is one in which R¹ and R² are as desired in the compound of formula I and M is an alkali or alkaline earth metal.

For example in step 1, a compound of formula (1), such as 3,4-dichloro-1,2,5-thiadiazole, is contacted with a metal amide. Appropriate metal amides may be obtained from commercial sources or are readily prepared by methods known to the skilled artisan. The use of alkali metal amides is preferred with lithium amides being more preferred. The reaction is carried out in a suitable non-reactive solvent or a mixture of non-reactive solvents, such as alkanes, for example, n-heptane or cyclohexane or ethers, for example, tetrahydrofuran, diethyl ether or tert-butylmethyl ether. The reaction is carried out with 1 to 3 molar equivalents of the compound of formula (1), with 1 to 2 molar equivalents being preferred and 1 molar equivalent being even more preferred. The reaction may be carried out by adding a solution of the compound of formula (1) to a solution of the metal amide or preferably by adding a solution of the metal amide to a solution of the compound of formula (1). Regardless of the order of addition, the reaction is carried out at a temperature of from about −78° C. to about 20° C. with a reaction temperature of about −78° C. to about −10° C. being preferred. The reaction generally requires 10 to 60 minutes. A compound of formula I may be used directly in the next reaction step without further purification or may be isolated and, if desired, purified.

In Scheme A, step 2, a compound of formula I is reacted with an appropriate nucleophile, Nu¹, to provide a compound of formula (3). It is understood that formula (3) encompasses, but is not limited to, compounds of formulas III, IV, and VII as described above. An appropriate nucleophile is one that gives a group V, R⁶, or A as described above or another group as desired in the final product. More specifically, a compound of formula I is reacted with a compound of the formula V—H where V is described above, a compound of formula R⁶—H where R⁶ is described above, a compound of the formula A-H where A is described above, or another nucleophile as desired. The reaction is conveniently carried out in a solvent in the presence of a suitable base. A suitable solvent for the reaction include the ether solvents with diethyl ether and tetrahydrofuran being preferred and tert-butylmethyl ether being more preferred. A suitable base includes the amines such as triethylamine and the metal alkoxides such as sodium tert-butoxide. The reaction is carried out with 0.5 to 2.0 molar equivalents of base with 0.75 to 1.5 molar equivalents being preferred and 0.75 molar equivalents more preferred. The reaction is carried out with 0.5 to 2 molar equivalents of a compound of formula I with 0.75 to 1.5 molar equivalents being preferred and 0.75 molar equivalent being more preferred. The reaction is carried out at a temperature of about −78° C. to about 20° C. with about −40° C. to about 20° C. being preferred. The reaction generally requires about one to eighteen hours. The product may be used directly in the next reaction step without further purification or may be isolated and, if desired, purified.

In Scheme A, step 3, a compound of formula (3) is reacted with another suitable nucleophile to give a compound of formula (4). It is understood that a compound of formula (4) encompasses, but is not limited to, compounds of formulas VI(a) and VI(b) as described above. An appropriate nucleophile, Nu² is one that is desired in the final product, and includes a nucleophile of the formula Nu¹. Examples of appropriate nucleophiles include a compound of formula H—V where V is defined above, a compound of formula H—R⁶ where R⁶ is as defined above, a compound of formula H-A where A is as defined above or another nucleophile as desired. The reaction is conveniently carried out in a solvent in the presence of a suitable base. A suitable base includes a metal alkoxide base with sodium tert-butoxide being preferred. The reaction is carried out with 0.1 to 1.0 molar equivalents of base with 0.1 to 0.5 molar equivalents being preferred and 0.1 molar equivalents more preferred. A suitable solvent for the reaction includes an ether solvent with diethyl ether and tetrahydrofuran being preferred, and tert-butylmethyl ether being more preferred. The reaction is carried out with 0.5 to 2 molar equivalents of a compound of formula (4) with 0.5 to 1.5 molar equivalents being preferred and 0.5 molar equivalents being more preferred. The reaction is carried out at a temperature of about −78° C. to about 20° C. with about −40° C. to about 20° C. being preferred and about 20° C. being even more preferred. The reaction generally requires about one to eighteen hours.

It is appreciated that the order of introduction of nucleophiles described in Scheme A can be reversed. More specifically, a nucleophile of formula Nu² can be introduced in step 2 to provide a compound of formula X and a nucleophile of formula Nu¹ can be introduced in step 3 to provide compounds of formula (4). A compound of formula X is depicted below.

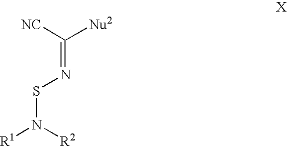

It is understood that a compound of formula X encompasses, but is not limited to, compounds of formulas III, IV and VII as described above.

The present invention is further illustrated by the following examples. These examples are illustrative only and are not intended to limit the invention in any way.

The terms used in the examples and preparations have their normal meanings unless otherwise designated. For example, “° C.” refers to degrees Celsius; “g” refers to gram or grams; “brine” refers to a saturated aqueous sodium chloride solution; “CH₂Cl₂” or “DCM” refers to dichloromethane; “DCE” refers to dichloroethane; etc. Chemical shifts are give in 8 and NMR spectra were obtained in CDCl₃, unless otherwise indicated.

### EXAMPLE 1

### Synthesis of 2-chloro-2-(N,N-diisopropylaminothio)imino acetonitrile

A solution of lithium diisopropylamide (4.5 mL of a 2M THF solution, 9 mmol) in 10 mL of anhydrous THF is added dropwise at −78° C. to a solution of 3,4-dichloro-1,2,5-thiadiazole (1.55 g, 10 mmol) in 10 mL of anhydrous THF. The cold reaction mixture is carried on directly to the next step. A sample of the reaction mixture is heated to 20° C., evaporated to dryness and purified by filtration on silica eluting with cyclohexane to afford an analytical sample of the title compound: ¹H-NMR (CDCl₃): δ (ppm) 1.2 (d, 12H), 3.5 (m, 2H); ¹³C-NMR (CDCl₃): δ (ppm) 100.3, 111.5; IR (cm⁻¹): 2229 (CN).

### EXAMPLE 2

### Synthesis of 2-(4-methylbenzylthio)2-(N,N-diisopropylaminothio)imino acetonitrile

The cold reaction mixture of 2-chloro-2-(N,N-diisopropylaminothio)imino acetonitrile of Example 1 is directly treated with a solution of 4-methylbenzyl mercaptan (1.25 g, 9 mmol) and triethylamine (1.019 g, 10 mmol) in 3 mL THF at −78° C. After 15 minutes, the solution is heated to room temperature for 1 hour, quenched with 20 mL water, extracted with 2×100 mL diethylether, dried over magnesium sulfate and evaporated to dryness. The residue is purified by flash chromatography on silica eluting with cyclohexane to afford 0.65 g (25% over 2 steps) of the title compound: HR-MS (DEI) m/z: found. 321.1332 (M^{+.}), Calcd for C₁₆H₂₃N₃S₂: 321.1333; ¹H-NMR (CDCl₃): δ (ppm) 1.17 (d, 12H), 2.35 (s, 3H), 3.47 (m, 2H), 4.37 (s, 2H); 7.14 (d, 2H), 7.27 (d, 2H); ¹³C-NMR (CDCl₃): δ (ppm) 20.9, 23.2, 37.9, 56.4, 110.7, 122.0; 128.8, 129.4, 132.0, 137.0; IR (cm⁻¹): 2216 (CN).

### EXAMPLE 3

### Synthesis of 2-(1-propylthio)-2-(N,N-diisopropylaminothio)imino acetonitrile

The title compound is prepared essentially as described in Example 2 employing propyl mercaptan. HR-MS (DEI) m/z: found. 259.1182 (M^{-.}), calcd for C₁₁H₂₁N₃S₂: 259.1177; ¹H-NMR (CDCl₃): δ (ppm) 1.07 (t, 3H), 1.20 (d, 12H), 1.80 (m, 2H), 3.17 (t, 2H), 3.5 (m, 2H); ¹³C-NMR (CDCl₃): δ (ppm) 12.9, 22.8, 23.5, 35.9, 56.4, 110.5, 122.9; IR (cm⁻¹): 2218 (CN).

### EXAMPLE 4

### Synthesis of 2-chloro-2-(N,N-bis(trimethylsilyl)aminothio)imino acetonitrile

A commercial 1M solution of lithium bis(trimethylsilyl)amide in THF/cyclohexane (100 ml, 100 mmol) is added dropwise over 1 hour to a solution of 3,4-dichloro-1,2,5-thiadiazole (23.25 g, 150 mmol) in diethylether (300 mL) at −78° C. After the addition is completed, water (100 mL) is slowly added and the reaction mixture is allowed to warm to room temperature. The organic layer is decanted and the aqueous layer is extracted with diethylether (2×100 mL). The combined organic layers are dried over magnesium sulfate and concentrated to dryness. The residue is purified by distillation under vacuum (70-74° C./0.3 mbar) to afford the title compound (20.5 g, 73%) as a pale yellow solid: m.p.=29-30° C.; ¹H-NMR (CDCl₃): δ (ppm) 0.2 (s, 18H); ¹³C-NMR (CDCl₃): δ (Ppm) 1.8, 99.4, 111.7, IR (cm⁻¹): 2227 (CN).

### EXAMPLE 5

### Synthesis of 2-chloro-2-(2,2,6,6-tetramethyl-1-piperidinylthio)imino acetonitrile

A commercial 2.5M n-BuLi solution (71 mL, 177 mmol) is added dropwise to a solution of 2,2,6,6-tetramethylpiperidine (25 g, 177 mmol) in anhydrous tert-butyl methyl ether (100 mL) at −20° C. The above solution is transferred into an addition funnel and is added dropwise to a solution of 3,4-dichloro-1,2,5-thiadiazole (27.4 g, 177 mmol) in anhydrous tert-butyl methyl ether (270 mL) at −50° C. Water (200 mL) is added and the reaction is allowed to warm to rt. The organic layer is decanted and the aqueous layer is extracted with diethyl ether (250 mL). The combined organic layers are washed with a 10% aqueous solution of acetic acid (100 mL) and water (100 mL). Toluene is added and the solution is concentrated under vacuum to afford the title compound (34 g, 74%): ¹H-NMR (CDCl₃): δ (ppm) 1.32 (s, 12H); 1.54-1.64 (m, 6H); ¹³C-NMR (CDCl₃): δ (ppm) 17.2, 27.5, 32.7, 41.0, 61.2, 100.0, 111.8; IR (cm⁻¹): 2225 (CN).

### EXAMPLE 6

### Synthesis of 2-chloro-2-(3,3,5,5-tetramethyl-4-morpholinylthio)imino acetonitrile

The title compound is prepared essentially as described in Example 5 employing 3,3,5,5-tetramethylmorpholine. ¹H-NMR (CDCl₃): δ (ppm) 1.23 (s, br, 6H), 1.41 (s, br, 6H), 3.40-3.58 (d, br, 4H), ¹³C-NMR (CDCl₃): δ (ppm) 25.6, 27.1, 59.7, 78.9, 101.4, 111.6; IR (cm⁻¹): 2231 (CN).

### EXAMPLE 7

### Synthesis of 2-chloro-2-(4-morpholinylthio)imino acetonitrile

The title compound is prepared essentially as described in Example 5 employing morpholine. ¹H-NMR (CDCl₃): δ (ppm) 3.22 (t, 4H), 3.75 (t, 4H); ¹³C-NMR (CDCl₃): δ (ppm) 54.9, 66.9, 104.5, 111.1; IR (cm⁻¹): 2236 (CN).

### EXAMPLE 8

### Synthesis of 2-chloro-2-(4-thiomorpholinylthio)imino acetonitrile

The title compound is prepared essentially as described in Example 5 employing thiomorpholine. ¹H-NMR (CDCl₃): δ (ppm) 2.72 (m, 4H), 3.50 (m, 4H); ¹³C-NMR (CDCl₃): δ (ppm) 28.1, 57.8, 104.1, 111.3; IR (cm⁻¹): 2229 (CN).

### EXAMPLE 9

### Synthesis of 2-chloro-2-(4-methyl-1-piperazinylthio)imino acetonitrile

The title compound is prepared essentially as described in Example 5 employing 1-methylpiperazine. HR-MS (DEI) m/z: found. 218.0383 (M^{+.}), calcd for C₇H₁ClN₄S: 218.0393; IR (cm⁻¹): 2215 (CN).

### EXAMPLE 10

### Synthesis of 2-chloro-2-(2,2,5,5-tetramethyl-2,5-disila-1-azacyclopentan-1-yl-thio)imino acetonitrile

The title compound is prepared essentially as described in Example 5 employing 2,2,5,5-tetramethyl-2,5-disila-1-azacyclopentane.

¹H-NMR (CDCl₃): δ (ppm) 0.20 (s, br, 12H), 0.87 (s, br, 4H); ¹³C-NMR (CDCl₃): δ (ppm) 0.4, 8.5, 100.0, 112.4; IR (cm⁻¹): 2222 (CN).

### EXAMPLE 11

### Synthesis of 2-chloro-2-(N,N-bis(diphenylmethylsilyl)aminothio)imino acetonitrile

The title compound is prepared essentially as described in Example 5 employing N,N-bis(diphenylmethylsilyl)amine. ¹H-NMR (CDCl₃): δ (ppm) 0.50 (s, br, 6H), 7.42-7.53 (m, 20H).

### EXAMPLE 12

### Synthesis of 2-chloro-2-(N,N-benzyl(trimethylsilyl)aminothio)imino acetonitrile

The title compound is prepared essentially as described in Example 5 employing N,N-benzyl(trimethylsilyl)amine.

HR-MS (DEI) m/z: found. 297.0525 (M^{+.}), calcd for C₁₂H₁₆ClN₃SSi: 297.0523

### EXAMPLE 13

### Synthesis of 2-chloro-2-(N-benzylaminothio)imino acetonitrile

The title compound is prepared essentially as described in Example 5 employing benzylamine. HR-MS (DEI) m/z: found. 225.0138 (M^{+.}), calcd for C₉H₈ClN₃S: 225.0127; IR (cm⁻¹): 2230 (CN).

### EXAMPLE 14

### Synthesis of 2-chloro-2-(N,N-bis(dimethylphenylsilyl)aminothio)imino acetonitrile

The title compound is prepared essentially as described in Example 5 employing N,N-bis(dimethylphenylsilyl)amine. ¹H-NMR (CDCl₃): δ (ppm) 0.50 (s, br, 12H), 7.42-7.53 (m, 10H); ¹³C-NMR (CDCl₃): δ (ppm) 76.5, 77.01, 77.52, 99.94, 111.65, 127.43, 127.52, 127.76, 129.79, 133.39, 133.84, 134.12, 137.17; IR (cm⁻¹): 2236 (CN); MS (GC-EI) m/z: 404 (MH^{+.}).

### EXAMPLE 15

### Synthesis of 2-(4-methylbenzylthio)-2-(N,N-bis(trimethylsilyl)aminothio)imino acetonitrile

### Method A

Triethylamine (1.5 mL, 10 mmol) and 4-methylbenzyl mercaptan (1.17 g, 8.5 mmol) are added to a solution of 2-chloro-2-(N,N-bis(trimethylsilyl)aminothio)imino acetonitrile (2.7 g, 9 mmol) in THF (20 mL) at rt. After 1 hour (the reaction is monitored by TLC eluting with n-hexane/diethylether 96:4), water (50 mL) and diethylether (50 mL) are added, the organic layer is decanted, dried over magnesium sulfate and evaporated. The residue is purified by flash chromatography on silica eluting with n-hexane/diethylether 9(9:1 to 96:4) to afford the title compound (1.7 g, 53%): HR-MS (DEI) m/z: found. 381.1170 (M^{+.}), calcd for C₁₆H₂₇N₃S₂Si₂: 381.1185; ¹H-NMR (CDCl₃): δ (ppm) 0.22 (s, 18H), 2.35 (s, 3H), 4.38 (s, 2H), 7.15 (d, 2H), 7.27 (d, 2H); ¹³C-NMR (CDCl₃): δ (ppm) 1.7, 20.9, 38.2, 111.2, 120.6, 128.7, 129.4, 132.0, 137.0; IR (cm⁻¹): 2216 (CN).

### Method B

Compound 4-methylbenzyl mercaptan (0.28 ml; 2 mmol) and sodium tert-butoxide (2 mL of a 1M THF solution, 2 mmol) are added to a solution of 2-chloro-2-(N,N-bis(trimethylsilyl)aminothio)imino acetonitrile (0.56 g, 2 mmol) in tert-butyl methyl ether (8 mL) at 40° C. After 15 minutes, the reaction mixture is treated with water and diethylether, the organic layer is decanted, dried over magnesium sulfate and evaporated. The residue is purified by flash chromatography on silica eluting with n-hexane/diethylether (9:1 to 96:4) to afford the title compound (0.53 g, 74%).

### EXAMPLE 16

### Synthesis of 2-(n-propylthio)-2-(N,N-bis(trimethylsilyl)aminothio)imino acetonitrile

The title compound is prepared essentially as described in Example 15, Method A employing n-propyl mercaptan. HR-MS (DEI) m/z: found. 319.1019 (M^{+.}), calcd for C₁₁H₂₅N₃S₂Si₂: 319.1029; ¹H-NMR (CDCl₃): δ (ppm) 0.23 (s, br, 18H), 1.06 (t, 3H), 1.79 (m, 2H), 3.06 (t, 2H); ¹³C-NMR (CDCl₃): δ (ppm) 1.5, 12.9, 23.5, 36.2, 110.9, 121.6; IR (cm⁻¹): 2218 (CN).

### EXAMPLE 17

### Synthesis of 2-[1-azabicyclo[3.2.1]octan-6-oxy]-2-(N,N-bis(trimethylsilyl)-aminothio)imino acetonitrile

The title compound is prepared essentially as described in Example 15, Method B employing 1-azabicyclo[3.2.1]octan-6-ol. HR-MS (DEI) m/z: found. 370.1674 (M^{+.}), calcd for C₁₅H₃₀N₄OSSi₂: 370.1679.

### EXAMPLE 18

### Synthesis of 2-[benzyloxy]-2-(N,N-bis(trimethylsilyl)aminothio)imino acetonitrile

The title compound is prepared essentially as described in Example 15, Method B employing benzyl alcohol. Mg (GC-EI) m/z: 351 (M^{+.}); ¹H-NMR (CDCl₃): δ (ppm) 0.23 (s, 18H), 5.20 (s, 2H), 7.40 (m, 5H); ¹³C-NMR (CDCl₃): δ (ppm) 1.7, 71.1, 109.0, 123.2, 128.3, 128.7, 127.8, 134.8; IR (cm⁻¹): 2224 (CN).

### EXAMPLE 19

### Synthesis of 2-diphenylmethoxy-2-(N,N-bis(trimethylsilyl)aminothio)imino acetonitrile

The title compound is prepared essentially as described in Example 15, Method B employing diphenylmethanol. HR-MS (DEI) m/z: found. 427.1560 (M^{+.}), calcd for C₂₁H₂₉N₃OSSi₂: 427.1570; IR (cm⁻¹): 2231 (CN).

### EXAMPLE 20

### Synthesis of 2-tert-butoxy-2-(N,N-bis(trimethylsilyl)aminothio)imino acetonitrile

The title compound is prepared essentially as described in Example 15, Method B employing tert-butanol. HR-MS (DEI) m/z: found. 317.1407 (M^{+.}), calcd for C₁₂H₁₇N₃OSSi₂: 317.1413; IR (cm⁻¹): 2224 (CN).

### EXAMPLE 21

### Synthesis of 2-benzylamino-2-(N,N-bis(trimethylsilyl)aminothio)imino acetonitrile

The title compound is prepared essentially as described in Example 15, Method A employing benzylamine. MS (GC-EI) m/z: 350 (M^{+.}); ¹H-NMR (CDCl₃): δ (ppm) 0.23 (s, 18H), 4.41 (s, br, 1H), 4.51 (s, br, 2H), 7.28-7.42 (m, 5H); ¹³C-NMR (CDCl₃): δ (ppm) 1.8, 48.0, 110.5, 123.3, 127.3, 127.8, 128.7, 137.0; IR (cm⁻¹): 2237 (CN).

### EXAMPLE 22

### Synthesis of 2-(4-morpholinyl)-2-(N,N-bis(trimethylsilyl)aminothio)imino acetonitrile

The title compound is prepared essentially as described in Example 15, Method A employing morpholine. MS (GC-EI) m/z: 330 (M^{+.}); IR (cm⁻¹): 2221 (CN).

### EXAMPLE 23

### Synthesis of 2-(4-methylbenzylthio)-2-(4-morpholinylthio)imino acetonitrile

Compound 4-methylbenzyl mercaptan (0.21 ml, 1.5 mmol) and sodium tert-butoxide (dropwise 1 mL of a 1M THF solution, 1 mmol) are added to a solution of 2-chloro-2-(4-morpholinylaminothio)imino acetonitrile (0.212 g, 1 mmol) in tert-butyl methyl ether (5 mL) at −40° C. After 15 minutes, the reaction is worked-up by addition of water and decantation of the organic layer. The title compound is obtained by flash chromatography on silica (hexanes-diethylether): HR-MS (DEI) m/z: found. 307.0820 (M^{+.}), calcd for C₁₄H₁₇N₃OS₂: 307.0813; ¹H-NMR (CDCl₃): δ (ppm) 2.34 (s, 3H), 3.20 (m, 4H), 3.73 (m, 4H), 4.40 (s, 2H), 7.15 (d, 2H), 7.27 (d, 2H); ¹³C-NMR (CDCl₃): δ (ppm) 21.1, 38.4, 58.8, 67.2, 110.7, 125.4, 128.9, 129.6, 131.7, 138.0; IR (cm⁻¹): 2218 (CN).

### EXAMPLE 24

### Synthesis of 2-benzyloxy-2-(4-morpholinylthio)imino acetonitrile

The title compound is prepared essentially as described in Example 23 employing benzyl alcohol. MS (GC-EI) m/z: 277 (M^{+.}); ¹H-NMR (CDCl₃): δ (ppm) 3.27 (m, 4H), 3.71 (m, 4H), 5.23 (s, 2H), 7.38 (s, br, 5H); ¹³C-NMR (CDCl₃): δ (ppm) 54.9, 67.2, 71.6, 108.4, 125.6, 128.5, 128.7, 134.4; IR (cm⁻¹): 2226 (CN).

### EXAMPLE 25

### Synthesis of 2-(n-propylthio)-2-(4-morpholinylthio)imino acetonitrile

The title compound is prepared essentially as described in Example 23 employing n-propyl mercaptan. HR-MS (DEI) m/z: found. 245.0659 (M^{+.}), calcd for C₉H₁₅N₃OS₂: 245.0656813; ¹H-NMR (CDCl₃): δ (ppm) 1.05 (t, 3H), 1.79 (m, 2H), 3.18-3.21 (m, 6H), 3.73 (t, 4H); ¹³C-NMR (CDCl₃): δ (ppm) 12.9, 23.5, 36.4, 54.7, 67.1, 110.3, 126.2; IR (cm⁻¹): 2220 (CN).

### EXAMPLE 26

### Synthesis of 2-(4-morpholinyl)-2-((4-morpholinyl)thio)imino acetonitrile

The title compound is prepared essentially as described in Example 23. Morpholine (2 molar equivalents) is added to a solution of 2-chloro-2-((4-morpholinyl)thio)imino acetonitrile (0.212 g, 1 mmol) in tert-butyl methyl ether (5 mL) at −40° C. After 15 minutes, the reaction is warmed to rt for 2 hours and worked-up as described in Example 23. MS (GC-EI) m/z: 256 (M^{+.}); ¹H-NMR (CDCl₃): δ (ppm) 3.22 (m, 4H), 3.44 (m, 4H), 3.72 (m, 8H); ¹³C-NMR (CDCl₃): δ (ppm) 45.8, 55.1, 65.9, 67.1, 107.2, 127.6; IR (cm⁻¹): 2226 (CN).

### EXAMPLE 27

### Synthesis of 2-(4-methylbenzylthio)-2-((2,2,6,6-tetramethyl-1-piperidinyl)thio)imino acetonitrile

Compound 4-methylbenzyl mercaptan (0.21 ml, 1.5 mmol) and potassium tert-butoxide (dropwise 1 mL of a 1M THF solution, 1 mmol) are added to a solution of 2-chloro-2-((2,2,6,6-tetramethyl-1-piperidinyl)thio)imino acetonitrile (0.26 g, 1 mmol) in tert-butyl methyl ether (5 mL) at −40° C. After 15 minutes, the reaction is worked-up by addition of water and decantation of the organic layer. The title compound (0.3 g, 90%) is obtained by flash chromatography on silica (hexanes-diethylether): MS (GC-EI) m/z: 361 (M^{+.}); ¹H-NMR (CDCl₃): δ (ppm) 1.29 (m, 6H), 1.34 (m, 6H), 1.5-1.7 (m, 6H), 2.37 (s, 3H), 4.42 (s, 2H), 7.17 (d, 2H), 7.30 (d, 2H); ¹³C-NMR (CDCl₃): δ (ppm) 17.1, 21.3, 29.1, 31.3, 38.4, 40.7, 60.6, 111.3, 121.6, 128.8, 129.4, 132.0, 137.7; IR (cm⁻¹): 2215 (CN).

### EXAMPLE 28

### Synthesis of 2-(n-propylthio)-2-((2,2,6,6-tetramethyl-1-piperidinyl)thio)imino acetonitrile

The title compound is prepared essentially as described in Example 27 employing n-propyl mercaptan. HR-MS (DEI)) m/z: found. 299.1487 (M^{+.}), calcd for C₁₄H₁₅N₃S₂: 299.1489; ¹H-NMR (CDCl₃): δ (ppm) 1.06 (t, 3H), 1.29 (m, 6H), 1.34 (m, 6H), 1.50-1.70 (m, 6H), 1.79 (m, 2H), 3.17 (t, 2H); ¹³C-NMR (CDCl₃): δ (ppm) 13.2, 17.2, 23.6, 29.2, 31.5, 36.5, 14.0, 60.7, 111.1, 122.7; IR (cm⁻¹): 2216 (CN);

### EXAMPLE 29

### Synthesis of 2-benzyloxy-2-((2,2,6,6-tetramethyl-1-piperidinyl)thio)imino acetonitrile

The title compound is prepared essentially as described in Example 27 employing benzyl alcohol and potassium tert-butoxide in tert-butyl methylether. HR-MS (DEI) m/z: found. 331.1726 (M^{+.}), calcd for C₁₈H₂₅N₃OS: 331.1718; ¹H-NMR (CDCl₃): δ (ppm) 1.30 (s, 6H), 1.33 (s, 6H), 1.58-1.67 (m, 6H), 5.19 (s, 2H), 7.38 (m, 5H); ¹³C-NMR (CDCl₃): δ (ppm) 17.1, 28.7, 31.5, 40.6, 60.5, 70.5, 108.6, 123.6, 127.7, 128.3, 128.5, 134.9; IR (cm⁻¹): 2225 (CN).

### EXAMPLE 30

### Synthesis of 2-(1-piperidine)-2-((2,2,6,6-tetramethyl-1-piperidinyl)thio)imino acetonitrile

The title compound is prepared essentially as described in Example 27 employing piperidine. HR-MS (DEI) m/z: found. 308.2033 (M^{+.}), calcd for C₁₆H₂₈N₄S: 308.2035; ¹H-NMR (CDCl₃): δ (ppm) 1.31 (s, br, 6H), 1.35 (s, br, 6H), 1.50-1.70 (m, 12H), 3.15 (m, 4H); ¹³C-NMR (CDCl₃): δ (ppm) 17.5, 24.5, 25.3, 28.9, 32.3, 41.2, 46.7, 60.6, 107.9, 125.4; IR (cm⁻¹): 2217 (CN).

### EXAMPLE 31

### Synthesis of 2-benzyloxy-2-((3,3,5,5-tetramethyl-4-morpholinyl)thio)imino acetonitrile

Benzylalcohol (0.125 ml, 1.2 mmol) and potassium tert-butoxide (1 mL of a 1M THF solution, 1 mmol) are added dropwise to a solution of 2-chloro-2-((3,3,5,5-tetramethyl-4-morpholinyl)aminothio)imino acetonitrile (0.262 g, 1 mmol) in tert-butyl methyl ether (4 mL) at −40° C. After 15 minutes, the reaction is worked-up by addition of water and decantation of the organic layer. The title compound (0.27 g, 85%) is obtained by flash chromatography on silica (hexanes-diethylether): HR-MS (DEI) m/z: found. 333.1515 (M^{+.}), calcd for C₁₇H₂₃N₃O₂S: 333.1511; ¹H-NMR (CDCl₃): δ (ppm) 1.21 (s, 6H), 1.36 (s, 6H), 3.38-3.57 (dd, 4H), 5.18 (s, 2H), 7.38 (s, 5H); ¹³C-NMR (CDCl₃): δ (ppm) 27.0, 27.3, 59.4, 76.9, 79.0, 108.7, 124.5, 128.0, 128.8, 134.9; IR (cm⁻¹): 2225 (CN).

### EXAMPLE 32

### Synthesis of (5R,6R)-6-[4-(n-propylthio)-[1,2,5]-thiadiazol-3-yloxy]-1-aza-bicyclo-[3.2.1]octane.

The title compound is prepared essentially as described in Example 33 employing 1-azabicyclo[3.2.1]octan-6-ol. ¹H NMR (CDCl₃) δ 1.07 (3H, t), 1.72-2.08 (5H, m), 2.3 (1H, m), 3.06 (1H, m), 3.16-3.6 (7H, m), 4.23 (1H, m), 5.5 (1H, m).

### EXAMPLE 33

### Synthesis of (5R,6R)-6-[4-(4-methyl-benzylthio)-[1,2,5]-thiadiazol-3-yloxy]-1-aza-bicyclo[3.2.1]octane

The title compound is prepared essentially as described in Example 32 employing 2-(4-methylbenzylthio)-2-((2,2,6,6-tetramethyl-1-piperidinyl)aminothio)imino acetonitrile of Example 27 and 1-azabicyclo[3.2.1]octan-6-ol.

### EXAMPLE 34

### Synthesis of 3-(3-tert-butylamino-2-hydroxypropoxy)-4-(1-morpholinyl):[1,2,5]-thiadiazole

Compound 3-tert-butylamino-1,2-propanediaol (4 mmol) and potassium tert-butoxide (0.4 mL of a 1M THF solution, 0.4 mmol) are added to a solution of 2-(4-morpholinyl)-2-(N,N-bis(trimethylsilyl)aminothio)imino acetonitrile (2 mmol) in tert-butylmethyl ether (20 mL). Stir at room temperature and monitor the reaction for completion by thin layer chromatography. Water is added to quench the reaction and the organic layer is separated. The aqueous layer is extracted with tert-butyl methyl ether. The combined organic layers are dried over magnesium sulfate and evaporated to dryness. The residue is purified by flash chromatography to afford the title compound.

1. A compound of formula I 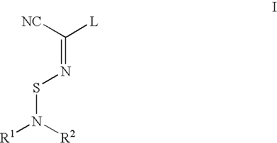
wherein
L is a leaving group;
R¹ and R² are independently selected from the group consisting of hydrogen, C1-C4 alkyl, benzyl optionally substituted with one or more substituents independently selected from the group consisting of halo, C1-C4 alkyl, C1-C4 alkoxy and —CF₃; and —SiRRR wherein each R is independently selected from the group consisting of C1-C4 alkyl, and phenyl optionally substituted with one or more substituents independently selected from the group consisting of C1-C4 alkyl, and C1-C4 alkoxy;
provided when R¹ is hydrogen then R² is not hydrogen;
or
R¹ and R² are taken together with the nitrogen in which they are attached to form a ring of formula II 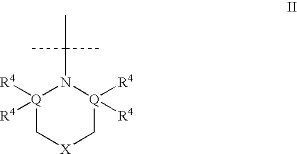
wherein
X is selected from the group consisting of bond, —CH₂—, —S(O)—ₙ, —O— and —NR²¹— wherein R²¹ is selected from the group consisting of C1-C4 alkyl, benzyl and phenyl optionally substituted with one or more substituents independently selected from the group consisting of halo, C1-C4 alkyl, and —CF₃;
n is 0, 1 or 2;
and
Q is selected from the group consisting of a bond, a carbon atom wherein each R⁴ is independently selected from the group consisting of hydrogen and C1-C4 alkyl; and a silicon atom wherein each R⁴ is independently selected from the group consisting of C1-C4 alkyl and phenyl.

2. A compound according to claim 1 wherein L is chloro.

3. A compound according to claim 1 or 2 wherein R¹ and R₂ are independently selected from the group consisting of hydrogen, C1-C4 alkyl, benzyl, and —SiRRR wherein each R is independently selected from the group consisting of C1-C4 alkyl and phenyl.

4. A compound according to claim 1 or 2 wherein R¹ and R² are taken together with the nitrogen in which they are attached to form a ring of formula II 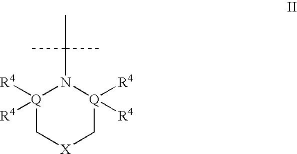
wherein
X is selected from the group consisting of bond, —CH₂—, —O— and —NR²¹— wherein R²¹ is selected from the group consisting of C1-C4 alkyl, benzyl and phenyl; and
Q is selected from the group consisting of a carbon atom wherein each R⁴ is independently selected from the group consisting of hydrogen and C1-C4 alkyl, and a silicon atom wherein each R⁴ is independently selected from the group consisting of C1-C4 alkyl and phenyl.

5. A compound according to claim 3 wherein R¹ and R² are independently selected from the group consisting of hydrogen, iso-propyl, trimethylsilyl, diphenylmethylsilyl, benzyl and dimethylphenylsilyl.

6. A compound according to claim 4 wherein a ring of formula II is selected from the group consisting of 2,2,6,6-tetramethyl-1-piperidinyl, 3,3,5,5-tetramethyl-4-morpholinyl, 4-morpholinyl, 4-thiomorpholinyl, 4-methyl-1-piperazinyl and 2,2,5,5-tetramethyl-2,5-disila-1-azacyclopentan-1-yl.

7. A compound according to claim 1 which is 2-chloro-2-(N,N-bis(trimethylsilyl)aminothio)imino acetonitrile.
